(19) United States
(12) Patent Application Publication
(10) Pub. No.: US 2021/0317528 A1
(43) Pub. Date: Oct. 14, 2021

(54) BLOOD TYPING USING DNA

(71) Applicant: GENOMICS USA, INC., ROUND ROCK, TX (US)

(72) Inventors: FREDERICK H. EGGERS, SAHUARITA, AZ (US); GEORGINA LOPEZ PADILLA, BURNABY (CA); PO LIN, TUSCON, AZ (US); KEVIN O'BRIEN, SAHUARITA, AZ (US); MICHAEL E. HOGAN, STONY BROOK, NY (US); KRISHNA JAYARAMAN, ROUND ROCK, TX (US)

(73) Assignee: GENOMICS USA, INC., ROUND ROCK, TX (US)

(21) Appl. No.: 17/250,447

(22) PCT Filed: Jul. 23, 2019
(86) PCT No.: PCT/US2019/042990

§ 371 Date: Jan. 22, 2021

Related U.S. Application Data
(60) Provisional application No. 62/701,942, filed on Jul. 23, 2018.

(51) Int. Cl. C12Q 1/6881; C12Q 1/6837; G16B 50/30; G16B 25/00

(52) U.S. Cl. CPC C12Q 1/6881; G16B 25/00; G16B 50/30; C12Q 1/6837

(57) ABSTRACT

Complex blood group typing can be performed at the DNA level, using for example, air-dried cheek swabs or finger prick blood in a microarray test that completely bypasses the need for DNA extraction prior to analysis of the blood group type.

### CROSS-REFERENCE TO RELATED APPLICATION

This application is a national stage filing of PCT/US2019/042990, filed Jul. 23, 2019; which claims the benefit of U.S. Provisional Application No. 62/701,942; filed Jul. 23, 2018, the entirety of both of which are hereby incorporated by reference.

### GOVERNMENT FUNDING

This invention was made with government support under grant 2 R44 HL110442 awarded by National Heart, Lung, Blood Institute (NHLBI). The government has certain rights in the invention.

### FIELD

The disclosure relates generally to blood typing. The disclosure relates specifically to blood typing using DNA.

### BACKGROUND

Serology-based blood group typing is a progenitor of personalized medicine (2). Recently, the genetic basis of blood group typing variation has become better understood. As a result, nearly all clinical applications of blood group typing could be converted from serology to DNA testing (3).

### SUMMARY

An embodiment of the disclosure is a microarray chip for performing blood group typing at a DNA level comprising a substrate; probes bound to the substrate; and a raw sample comprising DNA. In an embodiment, the raw sample is an air-dried cheek swab. In an embodiment, the raw sample is blood. In an embodiment, there are ABO-Rh probes. In an embodiment, the probe combination is such that a Rh-reaction will only occur if a Rh-deletion is present. In an embodiment, there are Weak D probes. In an embodiment, the probes are selected from SEQ ID NO: 163-180. In an embodiment, there are Minor Antigen probes. In an embodiment, the probes are selected from SEQ ID NO: 45-68 and SEQ ID NO: 105-126.

An embodiment of the disclosure is a method of performing blood group typing comprising obtaining a raw sample from an individual; amplifying a target sequence to obtain an amplified target sequence; labeling the amplified target sequence to obtain a labeled amplified target sequence; adding the labeled amplified target sequence to a microarray chip; hybridizing the labeled amplified target sequence to at least one probe present on the microarray chip; washing the microarray chip; and measuring fluorescence of the microarray chip. In an embodiment, the raw sample is an air-dried cheek swab. In an embodiment, the method further comprises preparing the air-dried cheek swab from the individual by a rapid 30 min soak in an aqueous release buffer; wherein amplification of blood group loci occurs by PCR from the soaking product; and wherein the labeling is with a fluorophore by PCR to generate single-stranded DNA. In an embodiment, the raw sample is blood.

An embodiment of the disclosure is a computer program for performing complex blood group typing at the DNA level utilizing the method; wherein the software is installed on a computer. In an embodiment, the computer is part of a scientific instrument. In an embodiment, the computer interacts with a scientific instrument.

An embodiment of the disclosure is a method of building a database of pre-qualified blood donors comprising providing registration information of an individual; providing a raw sample of the individual to a collection location; performing blood group typing on the raw sample; and adding the blood group typing to a database comprising the registration information of the individual. In an embodiment, the raw sample is a cheek swab sample. In an embodiment, the collection location is a laboratory. In an embodiment, the raw sample is mailed to the laboratory. In an embodiment, the database is searched for a desired blood group typing.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 depicts a comparison of a T-chip microarray and other DNA-based tests.

FIG. 2 depicts a process for coupling Raw Sample Genotyping (RSG) to low-cost T-chip microarray testing.

FIG. 3 depicts an ABO-Rh sub-assembly. FIG. 3A shows microarray hybridization probe locations and FIG. 3B shows primer design.

FIG. 4 depicts a typical T-chip microarray including T-chip microarray image data and automatic T-chip allelotype analysis.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3rd Edition.

The inventors have demonstrated that by coupling two technologies, “Raw Sample Genotyping” and “Low Cost Microarray Manufacture”, it is possible to perform complex blood group typing at the DNA level, on air-dried cheek swabs (or finger prick blood) as a microarray test. The test has been named the “T-Chip” and bypasses the need for DNA extraction prior to analysis of the blood group type. A focus is to deliver that ability to obtain complex blood group typing as a new type of molecular epidemiology. By analogy to well-known studies such as the National Marrow Donor Program (NMDP), a goal of this disclosure is to enable a very large donor population to be pre-screened at home, with a cheek swab, so that those donors would then stand ready to donate blood, much as NMDP volunteers are screened with a cheek swab to obtain their HLA-type for marrow donation (1).

A relatively small number of venous blood samples and cheek swabs from volunteers with a known blood type were tested. Only the principal blood types of clinical significance (ABO, Rh) were known for these samples and thus the highest level of technical refinement has been obtained for those more standard blood types. Additional work was completed for a number of minor blood types of secondary import, using synthetic gene (SG) fragments. The (SG) data indicate that the minor blood types can be analyzed directly from raw blood or raw cheek swabs in a way that bypasses DNA extraction.

It is now possible to obtain high resolution DNA-based blood typing in a clinic or blood bank via core-lab based sequencing and via hybridization-based analysis: using multiple qPCR tests (3), multiplexed solid state microarrays (5,6), or fluid-phase Luminex bead arrays (7). The potential value of both multiplexed microarray or Luminex testing has become highly attractive. Both platforms have been commercialized and are presently used by AABB certified blood banks and in some cases as the basis for clinical practice.

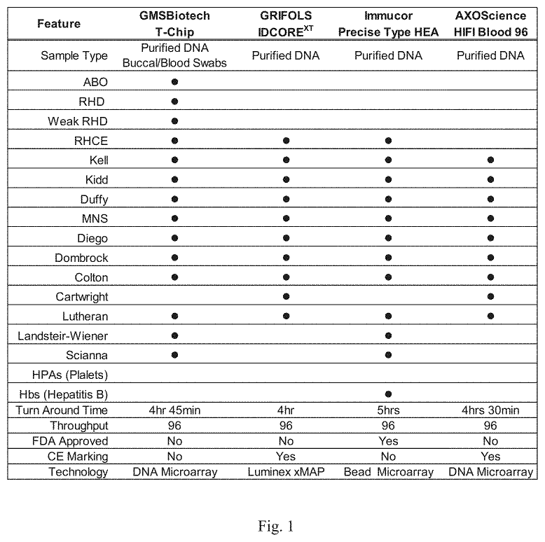

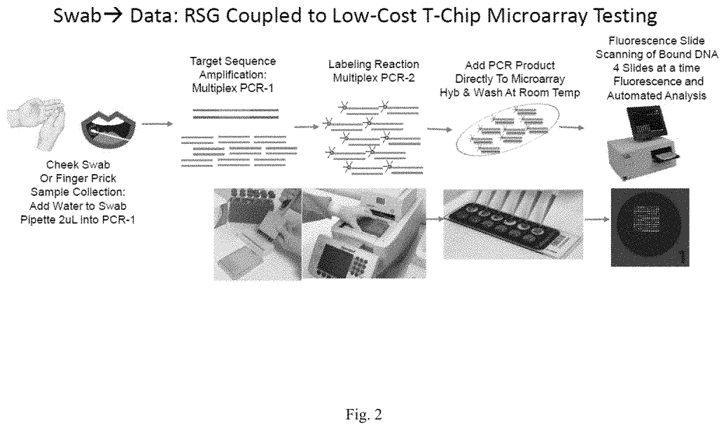

Two industry leaders are the Grifols ID CORExt test (Luminex based) and Immucor's Precise Type HEA (Bead-Chip microarray). These are compared to the T-Chip in FIG. 1.

The antigen coverage of all 4 tests is not the same. The T-chip test is more complete, measuring ABO, RHD and Weak D along with the minor antigens which are the primary focus of the other 3 DNA tests.

There are at least two differences between the T-Chip and the other three technologies. The T-Chip test supports a medical testing market that the other technologies cannot: namely, the deployment of DNA based blood group typing as the basis for Public Health Screening and Research Epidemiology of the Blood Group Type as a Disease Risk factor (8-10).

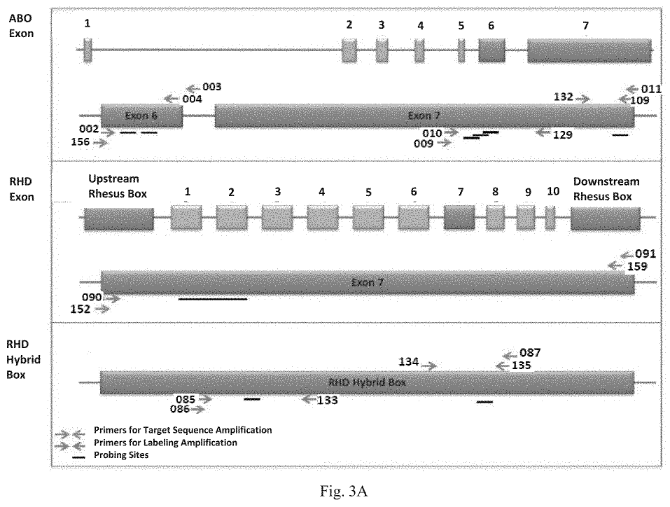

The first difference is Raw Sample Genotyping (RSG) enables low-cost field collection for blood group typing. RSG allows complex microarray testing to be performed on raw samples in the complete absence of DNA extraction and DNA characterization (11,12). Based on RSG, the T-Chip will be able to use cheek swabs (or a dried blood spot) as input for high throughput blood group typing. In an embodiment, using an inexpensive heat block, 100 swabs can be prepared simultaneously for T-Chip testing via a rapid 30-minute soak in an aqueous release buffer. In an embodiment, the soaking product is used, as-is, for PCR amplification of blood group loci, then labeled with a fluorophore (also by PCR) to generate single-stranded DNA that can be pipetted as-is from PCR tube and transferred without manipulation directly to the microarray (FIG. 2). The other tests (ID CORExt, Precise Type, HiFi) require DNA purification and characterization in their test workflow: which nearly doubles the labor, cost and time required to prepare samples for analysis, while also giving rise to significant DNA loss and dilution. Because of the DNA loss and dilution attendant to DNA purification, neither ID CORExt or Precise Type HEA or HiFi are qualified for swab-based collection, whereas the T-Chip test is optimized to exploit the use of such raw swabs. The ability to use ordinary cheek swabs, with little-to-no sample preparation will position the T-Chip test as a unique technology solution for swab based (epidemiological) field applications of DNA-based blood group typing.

The second difference is microarray technology enables low-cost microarray analysis for blood group typing. The microarray technology allows DNA microarrays of the complexity required for blood group typing (@350 probes) to be mass produced at a rate of several thousand arrays per day, at a cost per microarray that is roughly ¼th the price per test of the Luminex based (ID CORExt), or Bead Array (Precise type), or Plate Based Array (HiFi) test: thereby dropping test consumable cost by a factor of @5. In addition, the microarray technology allows DNA hybridization (which is the basis for all 4 tests in FIG. 1) to be performed at lab ambient temperature without the need for temperature control or fluidics other than a simple pipette tip. Via that simplification, the T-Chip test is performed without any specialized lab equipment: whereas the Grifols Test requires Luminex fluidics (@$100K) and the Immucor and AXO test each require a highly-specialized microfluidics delivery system (also @$100K). The only specialized equipment required for the T-Chip is a generic fluorescence scanner from Sensovation for a cost of <$20K). The resulting drop in reagent cost by @5-fold, while also dropping the cost of ancillary equipment by more than a factor of 3 is also an enabling aspect of the T-Chip technology. FIG. 2.

The T-Chip test could enable fundamentally new aspects to the clinical and research utility of blood banks. Namely, the ability to pre-screen a very large donor community via a combination of web-site registration, cheek swab sample collection, and mail-in sample transport to an AABB (formerly known as the American Association of Blood Banks) laboratory: where the T-Chip technology would allow hundreds of samples a day to be collected and processed to generate complex DNA blood group profiles, which could grow to become a large regional database of pre-qualified donors.

In an embodiment, a proposed model is for a very low cost, community-scale blood group database-building. Once developed and deployed, the goal for the T-Chip test is to support targeted blood unit delivery for clinical practice and to support research into the role of blood group marker variation as a biomarker for disease risk.

One technology utilized here is Raw Sample Genotyping “RSG” technology, comprising the “front-end” of the T-Chip test. Another technology teaches the “back-end” of the T-Chip test, namely mass-production of DNA microarrays which are not only low-cost but display sensitivity and specificity near the theoretical limit defined by nucleic acid biophysics.

Three different products which perform DNA-based blood group typing are already on the market, based on Luminex beads and two different kinds of microarray technology (5-7). They were developed to analyze purified DNA from a venous blood draw, and therefore were well-positioned to be a routine test in an AABB blood bank.

The T-Chip test will also work with purified DNA and would be a simpler, much less expensive, and more accurate option than the three products above.

In an embodiment, a focus is to create a completely new population-scale market for DNA-based blood group typing wherein blood group typing can be based on inexpensive swab-based sample collection, followed by the elimination of all DNA purification steps, and analysis on a microarray platform which is inexpensive enough to support DNA based typing at a cost that is about the same as DNA-based microbial testing. In an embodiment, the initial deployment of the T-Chip will be to enable very large-scale pre-qualification of potential blood donors. In an embodiment, the T-Chip could evolve to be used to support universal blood group typing at birth (on the same Guthrie cards used since 1962) or as the basis for national-scale blood group typing in resource-limited markets such as Africa and South America. Realistically, not one of the current sets of predicate tests could address those important public-health-scale markets because they are too expensive in terms of labor and consumables.

T-Chip Design Principles. In an embodiment, the T-Chip microarray will accommodate the DNA from a single unpurified cheek swab as sample input, under conditions where the resulting steps in the microarray test (e.g., hybridization, washing, and data analysis) can be executed at room temperature by any lab technician, without special expertise or equipment other than an inexpensive optical scanner.

In an embodiment, a version of the “watchmaker's” decomposition into “sub-assemblies” was utilized. In an embodiment, the full set of blood typing tests was resolved into 4 multiplex PCR reactions with cognate microarray probe design to go with each. In an embodiment, each PCR reaction converts 2 μL of a raw swab eluate into a sample that is ready for microarray testing. Thus, a single swab (which yields @30 μL of eluate) can support at least 3 repeats of the entire T-Chip test. Target gene sub-assembly decomposition is as follows (I) ABO-Rh, (II) Minor Allele Variants, Group #1, (III) Minor Allele Variants, Group #2 and Weak D: (i.e., those genetic changes generating a subtle change in the Rh+ serotype not related to overt Rh deletion).

ABO-Rh. Another focus is on the ABO-Rh sub-assembly. This focus is for at least three reasons: 1. The ABO-Rh grouping defines much of blood group typing as ordinarily deployed. 2. Although ABO analysis is a relatively simple SNP design problem, the Rh+/− genotype is an unusually complex analytical problem, in that most Rh-serotypes are derived from a @1 kb long block deletion within the Rh gene. A key requirement for such Rh+/− discrimination, especially in a heterozygote, is to convert the 1 kb block deletion into a positive microarray signal, rather than a simple loss of copy number. 3. The ABO-Rh problem is sufficiently difficult that the predicate tests did not include it, choosing to focus on the minor alleles only.

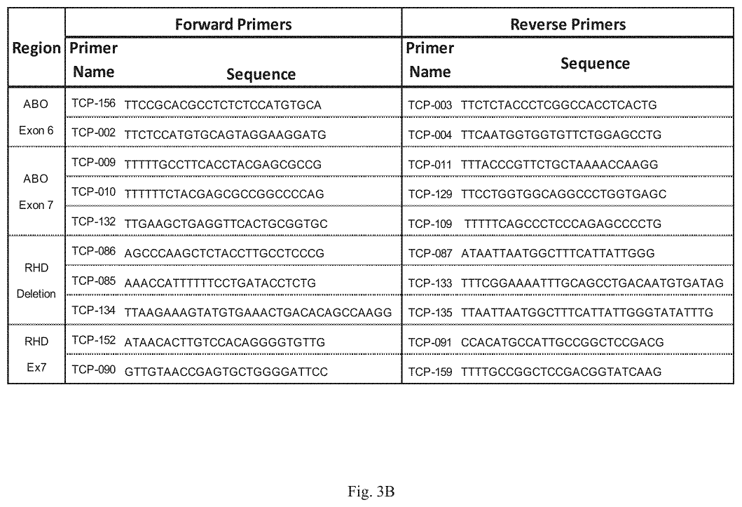

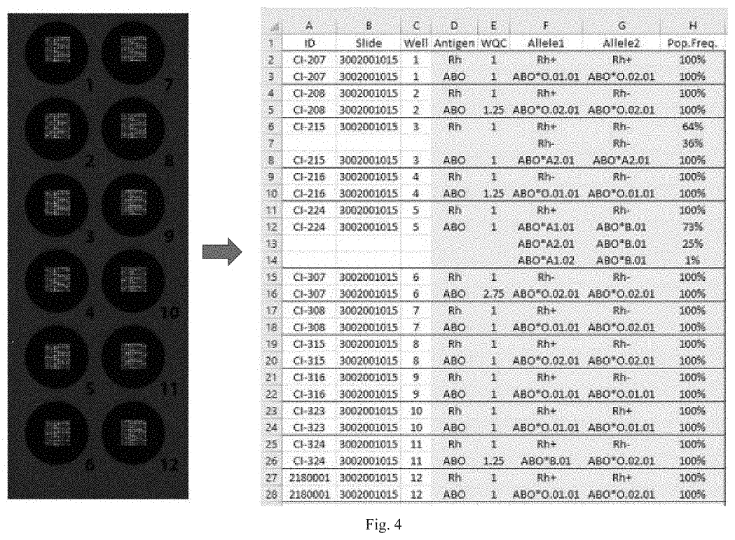

To convert the Rh-block deletion into a positive microarray signal, a PCR-microarray probe combination has been designed such that (Rh-) PCR reaction will only occur if the Rh-deletion is present. Consequently, the Rh-deletion creates two redundant microarray probe signals which only occur upon deletion: the result being that the Rh+/− heterozygote (obtained by the standard Rh-deletion) can now be unambiguously resolved, along with full ABO typing. FIG. 3. ABO-Rh Sub-Assembly: Primer Design & Microarray Hybridization Probe Locations.

The Minor Allele Variants #1, #2: In Tables I and II, the Minor Allele variants have been grouped into two sub-assemblies for the purpose of PCR amplification. For the first time, T-Chip microarray data for both sets (Tables III and IV), using custom made gene-sized DNA fragments (made by Synthetic Genetics Technology) are shown, which each present the known clinically relevant SNP changes (13).

TABLE I
Minor Blood Group #1, #2: Primer Design & Microarray Probe Locations. Probe and Primer Sequences
for Minor Allele Set #1

| | | | | PCR | | |
|---|---|---|---|---|---|---|
| | | | | Product | | Probe Specificity |
| | | PCR Primer | Primer sequence | size | ASO Probe sequence | (Analyte or Allele name) |
| Minor Antigen | Primary PCR | RHCE Exon 2 1′FP | TGTGGCCTTCAACCTCTTCATGCTG | 144 bp | AGTTCCCTCCTGG | 307C (RHCE*c) |
| Multiplex PCR | Primers | RHCE Exon 2 1′RP | (Seq Tag)AATACCTGAACAGTGTGATGACCAC | | | |
| Reaction 1 | Secondary | RHCE Exon 2 2′FP | TCTTCATGCTGGCGCTTGGTGTGCA | 130 bp | AGTTCCCTTCTGG | 307T (RHCE*C, RHD) |
| | PCR Primers | Universal Tag Primer CY3 | TTTTGACTAGGAAACAGCTATGACCATG | | | |
| | Primary PCR | RHCE Exon 5 1′FP | TCTTGTGGATGTTCTGGCCAAGTGT | 155 bp | TCAACTCTGCTCTG | 676G (RHCE*e) |
| | Primers | RHCE Exon 5 1′RP | (Seq Tag)CCCTGAGATGGCTGTCACCACACTG | | TCAACTCTCCTCTG | 676C (RHCE*E) |
| | Secondary | RHCE Exon 5 2′FP | TTGTGGATGTTCTGGCCAAGTGTCA | 153 bp | TATGCTCTAGCAGT | 733C |
| | PCR Primers | Universal Tag Primer CY3 | TTTTGACTAGGAAACAGCTATGACCATG | | TATGCTGTAGCAGT | 733G (VS) |
| | Primary PCR | Exon 7 1′FF | (Seq Tag)CTCCGTCATGCACTCCATCTTCAGC | 139 bp | GCAGACCCAGCA (AS) | 1006G |
| | Primers | RHCE Exon 7 1′RP | GACCCACATGCCATTGCCGTTCCAG | | | |
| | Secondary | Universal Tag Primer CY3 | TTTTGACTAGGAAACAGCTATGACCATG | 130 bp | GCAGACACAGCA (AS) | 1006T (V) |
| | PCR Primers | RHCE Exon 7 2′RP | GCCATTGCCGTTCCAGACAGTATGA | | | |
| | Primary PCR | KEL Exon 6 1′FP | CTTGGAGGCTGGCGCATCTCTGGTA | 120 bp | AACCGAACGCTGA | 578c (k) |
| | Primers | KEL Exon 6 1′RP | (Seq Tag)GAAATGGCCATACTGACTCATCAGA | | | |
| | Secondary | KEL Exon 6 2′FP | GAGGCTGGCGCATCTCTGGTAAATG | 116 bp | TAACCGAATGCTGA | 578T (K) |
| | PCR Prmmers | Universal Tag Prrmer CY3 | TITTGACTAGGAAACAGCTATGACCATG | | | |
| | Primary PCR | KEL Exon 8 1′FP | AGACCCAAGCAAGGTGCAAGAACAC | 133 bp | CACTTCACGGCT | 841C (Kp^{b}) |
| | Primers | KEL Exon 8 1′RP | (Seq Tag)TGCCCTGTGCCCGCCGCTGCTCCAG | | | |
| | Secondary | KEL Exon 8 2′FP | AGGTGCAAGAACACTCTTCCTTGTC | 122 bp | CACTTCATGGCTG | 841T (Kp^{a}) |
| | PCR Primers | Universal Tag Primer CY3 | TTTTGACTAGGAAACAGCTATGACCATG | | | |
| | Primary PCR | KEL Exon 17 1′FP | CCCTATGTTCTCTTGCTGTATGTTC | 141 bp | CTGCCTCGCCT | 1790T (Js^{b}) |
| | Primers | KEL Exon 17 1′RP | (Seq Tag)TTCAGGCACAGGTGAGCTTCCTGGA | | | |
| | Secondary | KEL Exon 17 2′FP | TTCTCTTGCTGTATGTTCTCTTGTC | 134 bp | CTGCCCCGCCT | 1790C (Js^{a}) |
| | PCR Primers | Universal Tag Primer CY3 | TTTTGACTAGGAAACAGCTATGACCATG | | | |
| | Primary PCR | Duffy Exon 2a 1′FP | (Seq Tag)GTGTGAATGATTCCTTCCCAGATG | 121 bp | TGGCATCATAGTCT (As) | 125A (Fy^{b}) |
| | Primers | Duffy Exon 2a 1′RP | CAGAGTCATCCAGCAGGTTACAGGA | | | |
| | Secondary | Universa1 Tag Primer CY3 | TTTTGACTAGGAAACAGCTATGACCATG | 114 bp | TGGCACCATAGTC (AS) | 125G (Fy^{a}) |
| | PCR Primers | Duffy Exon 2a 2′RP | ATCCAGCAGGTTACAGGAGTGGCAG | | | |
| | Primary PCR | Duffy Promotor 1′FP | CAGAACCTGATGGCCCTCATTAGTC | 97 bp | GCTCTTATCTTGGA | −67 (Fy^{b}) |
| | Primers | Duffy Promoter 1′RP | (Seq Tag)GGACGGCTGTCAGCGCCTGTGCT | | | |
| | Secondary | Duffy Promotor 2′FP | ACCTGATGGCCCTCATTAGTCCTTG | 93 bp | GCTCTTACCTTGG | −67 (FY*01N.01) |
| | PCR Primers | Universal Tag Primer CY3 | TTTTGACTAGGAAACAGCTATGACCATG | | | |
| | Primary PCR | Duffy Exon 2b 1′FP | GCTAGCAGCACTGTCCTCTTCATGC | 105 bp | CTCTTCCGCTGG | 265C (Fy^{b}) |
| | Primers | Duffy Exon 2b 1′RP | (Seq Tag)AGGACAGGCCAGCCAGGGCAGAGCT | | | |
| | Secondary | Duffy Exon 2b 2′FP | CACTGTCCTCTTCATGCTTTTCAGA | 97 bp | TCTCTTCTGCTGG | 265T FY*02M.01 |
| | PCR Primers | Universal Tag Primer CY3 | TTTTGACTAGGAAACAGCTATGACCATG | | | |
| | Primary PCR | Kidd Exon 9 1′FP | (Seq Tag)TCTTAACAGGACTCAGTCTTTCAGC | 138 bp | TAGATGTCCTCAAAT (AS) | 838G (Jk^{a}) |
| | Primers | Kidd Exon 9 1′RP | AGAGAGCTGTTGAAACCCCAGAGTC | | | |
| | Secondary | Universa1 Tag Primer CY3 | TTTTGACTAGGAAACAGCTATGACCATG | 132 bp | TAGATGTTCTCAAAT (AS) | 838A (JK^{b}) |
| | PCR Primers | Kidd Exon 9 2′RP | CTGTTGAAACCCCAGAGTCCAAAGT | | | |
| | Primary PCR | MNS GYPA Exon 2 1′FP | TTCTCAACTTCTATTTTATACAGCA | 183 bp | TCAGCATCAAGTAC | 59C (M) |
| | Primers | MNS GYPA Exon 2 2′RP | (Seq Tag)AGATGTAACTCTTTGTGACTGAAGA | | | |
| | Secondary | MNS GYPA Exon 2 2′FP | CTTCTATTTTATACAGCAATTGTGA | 119 bp | TCAGCATTAAGTAC | 59T (N) |
| | PCR Primers | Universal Tag Primer CY3 | TTTTGACTAGGAAACAGCTATGACCATG | | | |
| | | | SEQ ID NO: 1-44 | | SEQ ID NO: 45-68 | |

TABLE II
Minor Blood Group #1, #2: Primer Design & Microarray Probe Locations.
Probe and Primer Sequences for Minor Allele Set #2

| | | | | PCR | | |
|---|---|---|---|---|---|---|
| | | | | Product | | Probe Specificity |
| | | PCR Primer | Primer sequence | size | ASO Probe sequence | (Analyte or Allele name) |
| Minor Antigen | Primary PCR | MNS GYPB Exon 4 1′FP | AATGATTTTTTTCTTTGCACATGTC | 143 bp | GAGAAACGGGACA | 143C (s) |
| Multiplex PCR | Primers | MNS GYPB Exon 4 1′RP | (Seq Tag)AATATTAACATACCTGGTACAGTGA | | | |
| Reaction 2 | Secondary | MNS GYPB Exon 4 2′FP | TCTTTCTTATTTGGACTTACATTGA | 120 bp | GAGAAATGGGACAA | 143T (s) |
| | PCR Primers | Universal Tag | TTTTGACTAGGAAACAGCTATGACCATG | | | |
| | | Primer CY3 | | | | |
| | Primary PCR | MNS GYPB Exon 5 1′FP | (Seq Tag)TTATTTTGTGTGTGATGGCTGGTAT | 178 bp | GGATCGTTCCAATA (AS) | 230C (GYPB) |
| | Primers | MNS GYPB Intron 5 1′RP | AACTCAGAGGAATAAACCCTCCTAG | | GGATCATTCCAATAA (AS) | 230T (GYP*NY, GYP*He(NY)) |
| | Secondary | Universal Tag | TTTTGACTAGGAAACAGCTATGACCATG | 153 bp | CTGAATTCTCACCT (AS) | Intron5 G (GYPB) |
| | | Primer CY3 | | | | |
| | PCR Primers | MNS GYPB Intron 5 2′RP | AGCTGTTCACACTGGTATTTAGAGC | | CTGAATTATCACCTT (AS) | Intron5 T(GYP*NY, *He |
| | | | | | | (NY),*P2, *He(P2) |
| | Primary PCR | Lu Exon 3 1′FP | GGACACCCGGAGCTGAGAGCCTGCC | 123 bp | CCCCGCCTAGC | 230G (LU^{b}) |
| | Primers | Lu Exon 3 1′RP | (Seq Tag)GCTCAGAGCCCTGCATCTCAGCCGA | | | |
| | Secondary | Lu Exon 3 2′FP | CCCGCGCCCACAGACCGACCGCTCG | 100 bp | CCCCCACCTAGC | 230A (LU^{a}) |
| | PCR Primers | Universal Tag | TTTTGACTAGGAAACAGCTATGACCATG | | | |
| | | Primer CY3 | | | | |
| | Primary PCR | Di Exon 19 1′FP | GGCATCCAGATCATCTGCCTGGCAG | 127 bp | CACGCCGGCCT | 2561C (Di^{b}) |
| | Primers | Di Exon 19 1′RP | (Seq Tag)GCAGCGGCACAGTGAGGATGAGGAC | | | |
| | Secondary | Di Exon 19 2′FP | CAGATCATCTGCCTGGCAGTGCTGT | 121 bp | CACGCTGGCCT | 2561T (Di^{a}) |
| | PCR Primers | Universal Tag | TTTTGACTAGGAAACAGCTATGACCATG | | | |
| | | Primer CY3 | | | | |
| | Primary PCR | Co Exon 1 1′FP | (Seq Tag)CTGCCCTGGGCTTCAAATACCCGGT | 119 bp | GGACCGCCGTC (AS) | 134C (Co^{a}) |
| | Primers | Co Exon 1 1′RP | GATGCTCAGCCCGAAGGCCAGCGAC | | | |
| | Secondary | Universal Tag | TTTTGACTAGGAAACAGCTATGACCATG | 105 bp | GGACCACCGTCT (AS) | 134T (Co^{b}) |
| | | Primer CY3 | | | | |
| | PCR Primers | Co Exon 1 2′RP | AGGCCAGCGACACCTTCACGTTGTC | | | |
| | Primary PCR | Dom Exon 2 1′FP | (Seq Tag)GCTGTTTAAAGTTATAAATATGAGC | 123 bp | ACCAGTTTCCTCT (AS) | 793A (Do^{a}) |
| | Primers | Dom Exon 2 1′RP | AGCTGACAGTTATATGTGCTCAGGT | | | |
| | Secondary | Universal Tag | TTTTGACTAGGAAACAGCTATGACCATG | 113 bp | ACCAGTCTCCTCT (AS) | 793G (Do^{b}) |
| | | Primer CY3 | | | | |
| | PCR Primers | Dom Exon 2 2′RP | TATATGTGCTCAGGTTCCCAGTTGA | | | |
| | Primary PCR | Dom Exon 2 1′FP | AGAAGAATTATTTTAGGATGTGGCA | 142 bp | ACCAAGGAAAAGTT | 323G (Hy+) |
| | Primers | Dom Exon 2 1′RP | (Seq Tag)GTYTAAAACAAAATAGCCACAGCGT | | ACCAAGTAAAAGTTC | 323T (Hy−) |
| | Secondary | Dom Exon 2 2′FP | GGCAAAAAGCCCACTTAGCCTGGCT | 123 bp | ATGACTACCACACA | 350C Jo(a+) |
| | PCR Primers | Universal Tag | TTTTGACTAGGAAACAGCTATGACCATG | | ATGACTATCACACA | 350T Jo(a−) |
| | | Primer CY3 | | | | |
| | Primary PCR | Lw Exon 1 1′FP | (Seq Tag)CTGCGGCAAGGCAAGACGCTCAGAG | 131 bp | AGCAGCTGGTAAG (AS) | 299A (LW^{a}) |
| | Primers | Lw Exon 1 1′RP | GCGCAGGTCACGAGGCAGTGCGCGA | | | |
| | Secondary | Universal Tag | TTTTGACTAGGAAACAGCTATGACCATG | 118 bp | GCAGCCGGTAAG (AS) | 299G (LW^{b}) |
| | | Primer CY3 | | | | |
| | PCR Primers | Lw Exon 1 2′RP | GGCAGTGCGCGAGGGAGCTCCAGGC | | | |
| | Primary PCR | Sc Exon 4 1′FP | (Seq Tag)TTGGGCACAGCCGAGCTGCTCTGCC | 150 bp | ACCGTCCCGGG (AS) | 169G (Sc1) |
| | Primers | Sc Exon 4 1′RP | CATCCCGGAATATGTGAACAGCCTG | | | |
| | Secondary | Universal Tag | TTTTGACTAGGAAACAGCTATGACCATG | 133 bp | ACCGTCCTGGG (AS) | 169A (Sc2) |
| | | Primer CY3 | | | | |
| | PCR Primers | Sc Exon 4 2′RP | ACAGCCTGGGAGCGCTGCGGGAATG | | | |
| | | | SEQ ID NO: 69-104 | | SEQ ID NO: 105-126 | |

TABLE III
T-Chip Data: Minor Allele Set #1

| | | Hybridization Signal from | Hybridization Signal from | |
|---|---|---|---|---|
| | | Perfect Match Allele | Alternate Allele Target | Ratio |
| Probe/specificity | | Target(RFU) | (RFU) | PM:MM |
| RHCE*c | 307C | 65535 | 857 | 76:1 |
| RHCE*C, RHD | 307T | 65407 | 4290 | 15:1 |
| RHCE*e | 676G | 32860 | 6005 | 5:1 |
| RHCE*E | 676C | 65535 | 696 | 94:1 |
| RHCE | 733C | 1000 | 80 | 13:1 |
| RHCE*VS | 733G | 856 | 95 | 9:1 |
| RHCE | 1006G | 65535 | 447 | 147:1 |
| RHCE*V | 1006T | 59052 | 9635 | 6:1 |
| KEL k | 578C | 65535 | 1341 | 49:1 |
| KEL K | 578T | 35283 | 19325 | 2:1 |
| KEL Kp^{b} | 841C | 65535 | 795 | 82:1 |
| KEL Kp^{a} | 841T | 19417 | 1369 | 14:1 |
| KEL Js^{b} | 1790T | 65535 | 9360 | 7:1 |
| KEL Js^{a} | 1790C | 65535 | 5418 | 12:1 |
| Duffy Fy^{b} | 125A | 65535 | 6814 | 10:1 |
| Duffy Fy^{a} | 125G | 65535 | 4782 | 14:1 |
| Duffy Fy^{b} | −67T | 65535 | 5171 | 13:1 |
| Duffy (FY*01N.01) | −67C | 65535 | 935 | 70:1 |
| Duffy Fy^{b} | 265C | 65535 | 4835 | 14:1 |
| Duffy (FY*02M.01) | 265T | 46764 | 13947 | 3:1 |
| Kidd Jk^{a} | 838G | 58770 | 1051 | 56:1 |
| Kidd Jk^{b} | 838A | 51771 | 2278 | 23:1 |
| MNS M | 59C | 46418 | 381 | 122:1 |
| MNS N | 59T | 30045 | 1455 | 21:1 |

TABLE IV
T-Chip Data: Minor Allele Set #2

| | | Hybridization Signal from | Hybridization Signal from | |
|---|---|---|---|---|
| | | Perfect Match Allele | Alternate Allele Target | Ratio PM |
| Probe/specificity | | Target(RFU) | (RFU) | vs. MM |
| MNS s | 143C | 18275 | 294 | 62:1 |
| MNS S | 143T | 11960 | 323 | 37:1 |
| MNS | 230C | 18342 | 393 | 47:1 |
| MNS NY, HeNY | 230T | 22564 | 622 | 36:1 |
| MNS | IN5G | 11779 | 193 | 61:1 |
| (GYP*NY, *He(NY), | IN5T | 46230 | 545 | 85:1 |
| *P2, *He(P2) | | | | |
| Lu^{b} | 230G | 65535 | 18036 | 4:1 |
| Lu^{a} | 230A | 20429 | 234 | 87:1 |
| Dib | 2561C | 2606 | 116 | 22:1 |
| Dia | 2561T | 44652 | 6633 | 7:1 |
| Co^{a} | 134C | 16709 | 875 | 19:1 |
| Co^{b} | 134T | 7040 | 416 | 17:1 |
| Do^{a} | 793A | 35017 | 921 | 38:1 |
| Do^{b} | 793G | 49339 | 444 | 111:1 |
| Do (Hy+) | 323G | 20862 | 1053 | 20:1 |
| Do (Hy−) | 323T | 29129 | 713 | 41:1 |
| Jo (a+) | 350C | 54381 | 747 | 73:1 |
| Jo (a−) | 350T | 30492 | 3118 | 10:1 |
| Lw^{a} | 299A | 522 | 129 | 40:1 |
| Lw^{b} | 299G | 5494 | 113 | 49:1 |
| Sc1 | 169G | 1688 | 206 | 8:1 |
| Sc2 | 169A | 59902 | 4347 | 14:1 |

The data shown Tables III and IV are T-Chip hybridization data for both the Minor Allele #1 (Table III) and Minor Allele #2 (Table IV) sub-assemblies. Generally, the specificity is very high (match/single mismatch >10). However a small number of probes [Duffy FY*02M01, Lub, Dia, Sc1] show lower specificity in the 4-10 range, which is not acceptable. Work is in progress to increase the performance of that small number via probe shortening.

Weak D

Substantial effort has begun to suggest that the so called “Weak D” serology (i.e., serological phenotypes in-between Rh+& Rh-) should be complemented by genetic analysis to aid in early treatment of the neonate (14-17).

A new “Weak D” sub-assembly into the design of the T-Chip microarray is included based on analysis of the following set of markers: Weak D types 1, 2 and 3. All markers can be resolved via simple SNP analysis, at a level of complexity that is a bit simpler than Minor Antigen Sets #1 or #2 (Tables V and VI). As is the case for ABO-Rh, none of the 3 commercialized Predicate Tests can generate Weak D data (FIG. 1).

TABLE V
Weak D BLood Group: Primer Design & Microarray Probe Locations

| | | | | PCR | | |
|---|---|---|---|---|---|---|
| | | | | Product | | Probe Specificity |
| | | PCR Primer | Primer sequence | size | ASO Probe sequence | (Analyte or Allele name) |
| Weak D and Partial D | Primary PCR | RH* Exon 1 1′FP | TGCCTGGTGCTGGTGGAACCCCTGC | 83 bp | TGAGCTCTAAGTAC | 8C (RHD, RHCE) |
| Multiplex PCR | Primers | RH* Exon 1 1′RP | (Seq Tag)GGCAGGCAGCGCCGGACAGACCGC | | | |
| Reaction | Secondary | RH* 5′NCR 2′FP | TGGTGCTGGTGGAACCCCTGCACAG | 79 bp | TGAGCTGTAAGTAC | 8G (Weak D Type 3) |
| | PCR Primers | Universal Tag | TTTTGACTAGGAAACAGCTATGACCATG | | | |
| | | Primer CY3 | | | | |
| | Primary PCR | RHD Exon 2 1′FP | CGAGCAGTTGGCCAAGATCTGACCG | 81 bp | TGGCTTGGGCTT | 186G (RHD) |
| | Primers | RHD Exon 2 1′RP | (Seq Tag)CCAGCTGTGTCTCCGGAAACTCGAG | | | |
| | Secondary | RHD Exon 2 2′FP | AGTTGGCCAAGATCTGACCGTGATG | 76 bp | TGGCTTTGGCTTC | 186T (DIIIa, DIVa) |
| | PCR Primers | Universal Tag | TTTTGACTAGGAAACAGCTATGACCATG | | | |
| | | Primer CY3 | | | | |
| | Primary PCR | RHD Exon 3 1′FP | (Seq Tag)TGCTTTGTCGGTGCTGATCTCAGTG | 119 bp | AACTGCGCCAAGT(AS) | 410C (RHD) |
| | Primers | RHD Exon 3 1′RP | TTACTGATGACCATCCTCAGGTTGC | | | |
| | Secondary | Universal Tag | TTTTGACTAGGAAACAGCTATGACCATG | 109 bp | AACTGCACCAAGTT(AS) | 410T (DIIIa, DIVa) |
| | | Primer CY3 | | | | |
| | PCR Primers | RHD Exon 3 2′RP | CATCCTCAGGTTGCCTAAAGCTGTC | | | |
| | Primary PCR | RHD Exon 4 1′FP | (Seq Tag)CTACCCGAGGGAACGGAGGATAAAG | 122 bp | GTTGCTGTCTGAT (AS) | 602C ((+)RHD, DIVa), |
| | Primers | RH* Exon 4 lRP | AGCCATTCTGCTCAGCCCAAGTAGG | | | ((−)DVI) |
| | Secondary | Universal Tag | TTTTGACTAGGAAACAGCTATGACCATG | 81 bp | GTTGCTCTCTGAT (AS) | 602G (DIIIa, DAR) |
| | | Primer CY3 | | | | |
| | PCR Primers | RHD Exon 4 2′RP | CCCCACCTTGTCCTTACCCAGCATG | | | |
| | Primary PCR | RHD Exon 5 1′FP | CTTGTGGATGTTCTGGCCAAGTTTC | 91 bp | TCCAATCGAAAGGA | 697G ((+)RHD),((−)DVI) |
| | Primers | RH* Exon 5 1′RP | (Seq Tag)TACAGCATAGTAGGTGTTGAACACG | | | |
| | Secondary | RHD Exon 5 2′FP | TGTTCTGGCCAAGTTTCAACTCTGC | 83 bp | CCAATCCAAAGGAA | 697C DV type1 |
| | PCR Primers | Universal Tag | TTTTGACTAGGAAACAGCTATGACCATG | | CCAATCAAAAGGAA | 697A DV type5 |
| | | Primer CY3 | | | | |
| | Primary PCR | RH* Exon 6 1′FP | (Seq Tag)TTACCCACACGCTATTTCTTTGCAG | 179 bp | CTGTGCACATAAGT (AS) | 809T (RHD) |
| | Primers | RHD Exon 6 1′RP | TGTCTAGTTTCTTACCGGCAGGTAC | | | |
| | Secondary | Universal Tag | TTTTGACTAGGAAACAGCTATGACCATG | 165 bp | CTGTGCCCATAAG (AS) | 809G (Weak D Type1) |
| | | Primer CY3 | | | | |
| | PCR Primers | RHD Exon 6 2′RP | CGGCAGGTACTTGGCTCCCCCGACG | | | |
| | Primary PCR | RHD Exon 7 1′FP | ATTCCCCACAGCTCCATCATGGGCT | 113 bp | TGCTTGATACCGT | 1048G (RHD) |
| | Primers | RHD Exon 7 1′RP | (Seq Tag)CCCACATGCCATTGCCGGCTCCGAC | | | |
| | Secondary | RHD Exon 7 2′FP | CTCCATCATGGGCTACAACTTCAGC | 102 bp | GTGCTTCATACCG | 1048C (DIVa) |
| | PCR Primers | Universal Tag | TTTTGACTAGGAAACAGCTATGACCATG | | | |
| | | Primer CY3 | | | | |
| | Primary PCR | RH* Exon 8 1′FP | GGATTGGCTTCCAGGTCCTCCTCAG | 85 bp | CCATCGTGATAGCTCTC | (+)1136C (RHD) |
| | Primers | RHD Ex8 1136C 1′RP | (Seq Tag)CTGACCTGTCAGGAGACCAGACATG | | | |
| | Secondary | RH* Exon 8 2′FP | GGCTTCCAGGTCCTCCTCAGCATTG | 80 bp | | (−)(RHD 1136C absent (DAU) |
| | PCR Primers | Universal Tag | TTTTGACTAGGAAACAGCTATGACCATG | | | |
| | | Primer CY3 | | | | |
| | Primary PCR | RHD Exon 9 1′RP | AAGGATTTCTGTTGAGATACTGTCG | 151 bp | AAACAGGTTTGCTC | 1154G (RHD) |
| | Primers | RHD Exon 9 1′RP | (Seq Tag)CATGAGGTGCTTTCCATATTTTAAG | | | |
| | Secondary | RHD Exon 9 2′FP | TGTCGTTTTGACACACAATATTTCG | 131 bp | AAACAGCTTTGCTC | 1154C (Weak D type 2) |
| | PCR Primers | Universal Tag | TTTTGACTAGGAAACAGCTATGACCATG | | | |
| | | Primer CY3 | | | | |
| | | | SEQ ID NO: 127-162 | | SEQ ID NO: 163-180 | |

TABLE VI
T-Chip Microarray Manufacture and Consumable Kits

| | Discrete | Total Probes |
|---|---|---|
| | DNA Probes | (Triplicate) |
| Gene Loci | in T-Chip | in T-Chip |
| ABO, Rh | 20 | 60 |
| Weak D | 24 | 72 |
| Minor Antigens #1, #2 | 50 | 150 |
| Controls | 14 | 42 |
| Total | 108 | 324 |
| Number of | 4 | [ABO-Rh], |
| PCR reactions | | [Minor Antigens #1], |
| Per T-Chip Test | | [Minor Antigens #2], |
| | | [Weak D] |
| Array Manufacture | Q-Array 2 (Tucson, AZ) | 3240 |
| (In House) | 90 × 12 T-Chips | T-Chip Arrays/Week |
| | arrays per batch | |
| | Up to 3 batches per week | |
| Array Manufacture | MI (Huntsville AL) | 18,000 |
| (OEM) | 500 × 12 T-Chip | T-Chip Arrays/Week |
| | Arrays per batch | |
| | Up to 3 batches per week | |
| PCR Kit manufacture | 48 arrays per kit | 100 kits/week |
| (In House) | Including all wsrt-ware | 4800 array kits/week |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

1. https://bethematch.org
2. Schwarz H P, Dorner F. Br J Haematol. Karl Landsteiner and his major contributions to haematology. 2003 May; 121(4):556-65.
3. Fasano R M, Sullivan H C, Bray R A, Gebel H M, Meyer E K, Winkler A M, Josephson C D, Stowell S R, Sandy Duncan A, Roback J D. Genotyping Applications for Transplantation and Transfusion Management: The Emory Experience. Arch Pathol Lab Med. 2017 March; 141(3):329-340. doi: 10.5858/arpa.2016-0277-SA.
4. Wagner F F, Flegel W A, Bittner R, Döscher A. Molecular typing for blood group antigens within 40 min by direct polymerase chain reaction from plasma or serum. Br J Haematol. 2017 March; 176(5):814-821. doi: 10.1111/bjh.14469. Epub 2016 Dec. 19.
5. Lopez M, Apraiz I, Rubia M, Piedrabuena M, Azkarate M, Veldhuisen B, Vesga M A, Van Der Schoot, Puente F, Tejedor D. Performance evaluation study of ID CORE XT, a high throughput blood group genotyping platform. Blood Transfus. 2016 Nov. 25: 1-7. doi: 10.2450/2016.0146-16. [Epub ahead of print]
6. Boccoz S A,Le Goff G C, Mandon C A, Corgier B P, Blum L J, Marquette C A. Development and Validation of a Fully Automated Platform for Extended Blood Group Genotyping.J Mol Diagn. 2016 January; 18(1):144-52. doi: 10.1016/j.jmoldx.2015.09.002. Epub 2015 Nov. 29.
7. http://www.immucor.com/en-us/Products/Pages/PreciseType-HEA.aspx
8. Vasan S K, Rostgaard K, Majeed A, Ullum H, Titlestad K E, Pedersen O B, Erikstrup C, Nielsen K R, Melbye M, Nyrén O, Hjalgrim H, Edgren G. ABO Blood Group and Risk of Thromboembolic and Arterial Disease: A Study of 1.5 Million Blood Donors.Circulation. 2016 Apr. 12; 133(15):1449-57; discussion 1457. doi: 10.1161/CIRCULATIONAHA.115.017563. Epub 2016 Mar. 3.
9. Cozzi G D, Levinson R T, Toole H, Snyder M R, Deng A, Crispens M A, Khabele D, Beeghly-Fadiel A. Blood type, ABO genetic variants, and ovarian cancer survival. PLoS One. 2017 Apr. 27; 12(4):e0175119. doi: 10.1371/journal.pone.0175119. eCollection 2017.
10. Dean L. ABO Blood Group. In: Pratt V, McLeod H, Dean, Malheiro A, Rubinstein W, editors. Medical Genetics Summaries [Internet]. Bethesda (Md.): National Center for Biotechnology Information (US); 2012-2012 Oct. 1.
11. Hogan M E, Lopez G, May M R, Abalos A T, Eggers F H, Obrien K M. Methods of PCR and HLA-Typing Using Unpurified Samples. U.S. Pat. No. 9,416,419. Issued Aug. 16, 2016.
12. Hogan M E, Lopez G, May M R, Abalos A T, Eggers F H, Obrien K M. Methods of PCR and HLA-Typing Using Raw Blood. U.S. Pat. No. 8,771,951. Issued Jul. 18, 2014.
13. https://www.idtdna.com/pages/products/genes/
14. Sandler S G, Chen L N, Flegel W A. Serological weak D phenotypes: a review and guidance for interpreting the RhD blood type using the RHD genotype. Br J Haematol. 2017 May 16. doi: 10.1111/bjh.14757. [Epub ahead of print]
15. Daniels G. Variants of RhD-current testing and clinical consequences. Br J Haematol. 2013 May; 161(4):461-70. doi: 10.1111/bjh.12275. Epub 2013 Feb. 25
16. Westhoff C M, Vege S, Hipsky C H, Horn T, Hue-Roye K, Keller J, Velliquette R, Lomas-Francis C, Chou S T, Reid M E. RHCE*ceAG (254C>G, Ala85Gly) is prevalent in blacks, encodes a partial ce-phenotype, and is associated with discordant RHD zygosity. Transfusion. 2015 November; 55(11):2624-32. doi: 10.1111/trf 13225. Epub 2015 Jul. 14.
17. Kacker S, Vassallo R, Keller M A, Westhoff CM, Frick K D, Sandler S G, Tobian A A. Financial implications of RHD genotyping of pregnant women with a serologic weak D phenotype. Transfusion. 2015 September; 55(9):2095-103. doi: 10.1111/trf.13074. Epub 2015 Mar. 21.
18. U.S. Pat. No. 9,416,419.
19. U.S. Pat. No. 8,771,951.
20. U.S. Pat. No. 8,575,325.

What is claimed is:

1. A microarray chip for performing blood group typing at a DNA level comprising
a substrate;
probes bound to the substrate; and
a raw sample comprising DNA.

2. The microarray chip of claim 1 wherein the raw sample is an air-dried cheek swab.

3. The microarray chip of claim 1 wherein the raw sample is blood.

4. The microarray chip of claim 1 wherein there are ABO-Rh probes.

5. The microarray chip of claim 4 wherein the probe combination is such that a Rh-reaction will only occur if a Rh-deletion is present.

6. The microarray chip of claim 1 wherein there are Weak D probes.

7. The microarray chip of claim 6 wherein the probes are selected from SEQ ID NO: 163-180.

8. The microarray chip of claim 1 where there are Minor Antigen probes.

9. The microarray chip of claim 8 wherein the probes are selected from SEQ ID NO: 45-68 and SEQ ID NO: 105-126.

10. A method of performing blood group typing comprising obtaining a raw sample from an individual;
amplifying a target sequence to obtain an amplified target sequence;
labeling the amplified target sequence to obtain a labeled amplified target sequence;
adding the labeled amplified target sequence to a microarray chip;
hybridizing the labeled amplified target sequence to at least one probe present on the microarray chip;
washing the microarray chip; and
measuring fluorescence of the microarray chip.

11. The method of claim 10 further comprising preparing an air-dried cheek swab from the individual by a rapid 30 min soak in an aqueous release buffer;
wherein amplification of blood group loci occurs by PCR from the soaking product; and
wherein the labeling is with a fluorophore by PCR to generate single-stranded DNA.

12. The method of claim 10 wherein the raw sample is blood.

13. A computer program for performing complex blood group typing at the DNA level utilizing the method of claim 10; wherein the software is installed on a computer.

14. The computer program of claim 13, wherein the computer is part of a scientific instrument.

15. The computer program of claim 13, wherein the computer interacts with a scientific instrument.

16. A method of building a database of pre-qualified blood donors comprising
providing registration information of an individual;
providing a raw sample of the individual to a collection location;
performing blood group typing on the raw sample; and
adding the blood group typing to a database comprising the registration information of the individual.

17. The method of claim 16 wherein the raw sample is a cheek swab sample.

18. The method of claim 16 wherein the collection location is a laboratory.

19. The method of claim 18 wherein the raw sample is mailed to the laboratory.

20. The method of claim 16 wherein the database is searched for a desired blood group typing.


SEQUENCE LISTING (200 sequences)

SEQ ID NO: 1 - DNA, 25 bp, Artificial Sequence
  Primers/Probes Sequences
tgtggccttcaacctcttcatgctg

SEQ ID NO: 2 - DNA, 25 bp, Artificial Sequence
  Primers/Probes Sequences
aatacctgaacagtgtgatgaccac

SEQ ID NO: 3 - DNA, 25 bp, Artificial Sequence
  Primers/Probes Sequences
tcttcatgctggcgcttggtgtgca

SEQ ID NO: 4 - DNA, 28 bp, Artificial Sequence
  Primers/Probes Sequences
ttttgactaggaaacagctatgaccatg

SEQ ID NO: 5 - DNA, 25 bp, Artificial Sequence
  Primers/Probes Sequences
tcttgtggatgttctggccaagtgt

SEQ ID NO: 6 - DNA, 25 bp, Artificial Sequence
  Primers/Probes Sequences
ccctgagatggctgtcaccacactg

SEQ ID NO: 7 - DNA, 25 bp, Artificial Sequence
  Primers/Probes Sequences
ttgtggatgttctggccaagtgtca

SEQ ID NO: 8 - DNA, 28 bp, Artificial Sequence
  Primers/Probes Sequences
ttttgactaggaaacagctatgaccatg

SEQ ID NO: 9 - DNA, 25 bp, Artificial Sequence
  Primers/Probes Sequences
ctccgtcatgcactccatcttcagc

SEQ ID NO: 10 - DNA, 25 bp, Artificial Sequence
  Primers/Probes Sequences
gacccacatgccattgccgttccag

SEQ ID NO: 11 - DNA, 28 bp, Artificial Sequence
  Primers/Probes Sequences
ttttgactaggaaacagctatgaccatg

SEQ ID NO: 12 - DNA, 25 bp, Artificial Sequence
  Primers/Probes Sequences
gccattgccgttccagacagtatga

SEQ ID NO: 13 - DNA, 25 bp, Artificial Sequence
  Primers/Probes Sequences
cttggaggctggcgcatctctggta

SEQ ID NO: 14 - DNA, 25 bp, Artificial Sequence
  Primers/Probes Sequences
gaaatggccatactgactcatcaga

SEQ ID NO: 15 - DNA, 25 bp, Artificial Sequence
  Primers/Probes Sequences
gaggctggcgcatctctggtaaatg

SEQ ID NO: 16 - DNA, 28 bp, Artificial Sequence
  Primers/Probes Sequences
ttttgactaggaaacagctatgaccatg

SEQ ID NO: 17 - DNA, 25 bp, Artificial Sequence
  Primers/Probes Sequences
agacccaagcaaggtgcaagaacac

SEQ ID NO: 18 - DNA, 25 bp, Artificial Sequence
  Primers/Probes Sequences
tgccctgtgcccgccgctgctccag

SEQ ID NO: 19 - DNA, 25 bp, Artificial Sequence
  Primers/Probes Sequences
aggtgcaagaacactcttccttgtc

SEQ ID NO: 20 - DNA, 28 bp, Artificial Sequence
  Primers/Probes Sequences
ttttgactaggaaacagctatgaccatg

SEQ ID NO: 21 - DNA, 25 bp, Artificial Sequence
  Primers/Probes Sequences
ccctatgttctcttgctgtatgttc

SEQ ID NO: 22 - DNA, 25 bp, Artificial Sequence
  Primers/Probes Sequences
ttcaggcacaggtgagcttcctgga

SEQ ID NO: 23 - DNA, 25 bp, Artificial Sequence
  Primers/Probes Sequences
ttctcttgctgtatgttctcttgtc

SEQ ID NO: 24 - DNA, 28 bp, Artificial Sequence
  Primers/Probes Sequences
ttttgactaggaaacagctatgaccatg

SEQ ID NO: 25 - DNA, 24 bp, Artificial Sequence
  Primers/Probes Sequences
gtgtgaatgattccttcccagatg

SEQ ID NO: 26 - DNA, 25 bp, Artificial Sequence
  Primers/Probes Sequences
cagagtcatccagcaggttacagga

SEQ ID NO: 27 - DNA, 28 bp, Artificial Sequence
  Primers/Probes Sequences
ttttgactaggaaacagctatgaccatg

SEQ ID NO: 28 - DNA, 25 bp, Artificial Sequence
  Primers/Probes Sequences
atccagcaggttacaggagtggcag

SEQ ID NO: 29 - DNA, 25 bp, Artificial Sequence
  Primers/Probes Sequences
cagaacctgatggccctcattagtc

SEQ ID NO: 30 - DNA, 23 bp, Artificial Sequence
  Primers/Probes Sequences
ggacggctgtcagcgcctgtgct

SEQ ID NO: 31 - DNA, 25 bp, Artificial Sequence
  Primers/Probes Sequences
acctgatggccctcattagtccttg

SEQ ID NO: 32 - DNA, 28 bp, Artificial Sequence
  Primers/Probes Sequences
ttttgactaggaaacagctatgaccatg

SEQ ID NO: 33 - DNA, 25 bp, Artificial Sequence
  Primers/Probes Sequences
gctagcagcactgtcctcttcatgc

SEQ ID NO: 34 - DNA, 25 bp, Artificial Sequence
  Primers/Probes Sequences
aggacaggccagccagggcagagct

SEQ ID NO: 35 - DNA, 25 bp, Artificial Sequence
  Primers/Probes Sequences
cactgtcctcttcatgcttttcaga

SEQ ID NO: 36 - DNA, 28 bp, Artificial Sequence
  Primers/Probes Sequences
ttttgactaggaaacagctatgaccatg

SEQ ID NO: 37 - DNA, 25 bp, Artificial Sequence
  Primers/Probes Sequences
tcttaacaggactcagtctttcagc

SEQ ID NO: 38 - DNA, 25 bp, Artificial Sequence
  Primers/Probes Sequences
agagagctgttgaaaccccagagtc

SEQ ID NO: 39 - DNA, 28 bp, Artificial Sequence
  Primers/Probes Sequences
ttttgactaggaaacagctatgaccatg

SEQ ID NO: 40 - DNA, 25 bp, Artificial Sequence
  Primers/Probes Sequences
ctgttgaaaccccagagtccaaagt

SEQ ID NO: 41 - DNA, 25 bp, Artificial Sequence
  Primers/Probes Sequences
ttctcaacttctattttatacagca

SEQ ID NO: 42 - DNA, 25 bp, Artificial Sequence
  Primers/Probes Sequences
agatgtaactctttgtgactgaaga

SEQ ID NO: 43 - DNA, 25 bp, Artificial Sequence
  Primers/Probes Sequences
cttctattttatacagcaattgtga

SEQ ID NO: 44 - DNA, 28 bp, Artificial Sequence
  Primers/Probes Sequences
ttttgactaggaaacagctatgaccatg

SEQ ID NO: 45 - DNA, 13 bp, Artificial Sequence
  Primers/Probes Sequences
agttccctcctgg

SEQ ID NO: 46 - DNA, 13 bp, Artificial Sequence
  Primers/Probes Sequences
agttcccttctgg

SEQ ID NO: 47 - DNA, 14 bp, Artificial Sequence
  Primers/Probes Sequences
tcaactctgctctg

SEQ ID NO: 48 - DNA, 14 bp, Artificial Sequence
  Primers/Probes Sequences
tcaactctcctctg

SEQ ID NO: 49 - DNA, 14 bp, Artificial Sequence
  Primers/Probes Sequences
tatgctctagcagt

SEQ ID NO: 50 - DNA, 14 bp, Artificial Sequence
  Primers/Probes Sequences
tatgctgtagcagt

SEQ ID NO: 51 - DNA, 12 bp, Artificial Sequence
  Primers/Probes Sequences
gcagacccagca

SEQ ID NO: 52 - DNA, 12 bp, Artificial Sequence
  Primers/Probes Sequences
gcagacacagca

SEQ ID NO: 53 - DNA, 13 bp, Artificial Sequence
  Primers/Probes Sequences
aaccgaacgctga

SEQ ID NO: 54 - DNA, 14 bp, Artificial Sequence
  Primers/Probes Sequences
taaccgaatgctga

SEQ ID NO: 55 - DNA, 12 bp, Artificial Sequence
  Primers/Probes Sequences
cacttcacggct

SEQ ID NO: 56 - DNA, 13 bp, Artificial Sequence
  Primers/Probes Sequences
cacttcatggctg

SEQ ID NO: 57 - DNA, 11 bp, Artificial Sequence
  Primers/Probes Sequences
ctgcctcgcct

SEQ ID NO: 58 - DNA, 11 bp, Artificial Sequence
  Primers/Probes Sequences
ctgccccgcct

SEQ ID NO: 59 - DNA, 14 bp, Artificial Sequence
  Primers/Probes Sequences
tggcatcatagtct

SEQ ID NO: 60 - DNA, 13 bp, Artificial Sequence
  Primers/Probes Sequences
tggcaccatagtc

SEQ ID NO: 61 - DNA, 14 bp, Artificial Sequence
  Primers/Probes Sequences
gctcttatcttgga

SEQ ID NO: 62 - DNA, 13 bp, Artificial Sequence
  Primers/Probes Sequences
gctcttaccttgg

SEQ ID NO: 63 - DNA, 12 bp, Artificial Sequence
  Primers/Probes Sequences
ctcttccgctgg

SEQ ID NO: 64 - DNA, 13 bp, Artificial Sequence
  Primers/Probes Sequences
tctcttctgctgg

SEQ ID NO: 65 - DNA, 15 bp, Artificial Sequence
  Primers/Probes Sequences
tagatgtcctcaaat

SEQ ID NO: 66 - DNA, 15 bp, Artificial Sequence
  Primers/Probes Sequences
tagatgttctcaaat

SEQ ID NO: 67 - DNA, 14 bp, Artificial Sequence
  Primers/Probes Sequences
tcagcatcaagtac

SEQ ID NO: 68 - DNA, 14 bp, Artificial Sequence
  Primers/Probes Sequences
tcagcattaagtac

SEQ ID NO: 69 - DNA, 25 bp, Artificial Sequence
  Primer/Probe Sequences
aatgatttttttctttgcacatgtc

SEQ ID NO: 70 - DNA, 25 bp, Artificial Sequence
  Primer/Probe Sequences
aatattaacatacctggtacagtga

SEQ ID NO: 71 - DNA, 25 bp, Artificial Sequence
  Primer/Probe Sequences
tctttcttatttggacttacattga

SEQ ID NO: 72 - DNA, 28 bp, Artificial Sequence
  Primer/Probe Sequence
ttttgactaggaaacagctatgaccatg

SEQ ID NO: 73 - DNA, 24 bp, Artificial Sequence
  Primer/Probe Sequence
tattttgtgtgtgatggctggtat

SEQ ID NO: 74 - DNA, 25 bp, Artificial Sequence
  Primer/Probe Sequence
aactcagaggaataaaccctcctag

SEQ ID NO: 75 - DNA, 28 bp, Artificial Sequence
  Primer/Probe Sequence
ttttgactaggaaacagctatgaccatg

SEQ ID NO: 76 - DNA, 25 bp, Artificial Sequence
  Primer/Probe Sequence
agctgttcacactggtatttagagc

SEQ ID NO: 77 - DNA, 25 bp, Artificial Sequence
  Primers/Probes Sequences
ggacacccggagctgagagcctgcc

SEQ ID NO: 78 - DNA, 25 bp, Artificial Sequence
  Primers/Probes Sequences
gctcagagccctgcatctcagccga

SEQ ID NO: 79 - DNA, 25 bp, Artificial Sequence
  Primers/Probes Sequences
cccgcgcccacagaccgaccgctcg

SEQ ID NO: 80 - DNA, 28 bp, Artificial Sequence
  Primers/Probes Sequences
ttttgactaggaaacagctatgaccatg

SEQ ID NO: 81 - DNA, 25 bp, Artificial Sequence
  Primers/Probes Sequences
ggcatccagatcatctgcctggcag

SEQ ID NO: 82 - DNA, 25 bp, Artificial Sequence
  Primers/Probes Sequences
gcagcggcacagtgaggatgaggac

SEQ ID NO: 83 - DNA, 25 bp, Artificial Sequence
  Primers/Probes Sequences
cagatcatctgcctggcagtgctgt

SEQ ID NO: 84 - DNA, 28 bp, Artificial Sequence
  Primers/Probes Sequences
ttttgactaggaaacagctatgaccatg

SEQ ID NO: 85 - DNA, 25 bp, Artificial Sequence
  Primers/Probes Sequences
ctgccctgggcttcaaatacccggt

SEQ ID NO: 86 - DNA, 25 bp, Artificial Sequence
  Primers/Probes Sequences
gatgctcagcccgaaggccagcgac

SEQ ID NO: 87 - DNA, 28 bp, Artificial Sequence
  Primers/Probes Sequences
ttttgactaggaaacagctatgaccatg

SEQ ID NO: 88 - DNA, 25 bp, Artificial Sequence
  Primers/Probes Sequences
aggccagcgacaccttcacgttgtc

SEQ ID NO: 89 - DNA, 25 bp, Artificial Sequence
  Primers/Probes Sequences
gctgtttaaagttataaatatgagc

SEQ ID NO: 90 - DNA, 25 bp, Artificial Sequence
  Primers/Probes Sequences
agctgacagttatatgtgctcaggt

SEQ ID NO: 91 - DNA, 28 bp, Artificial Sequence
  Primers/Probes Sequences
ttttgactaggaaacagctatgaccatg

SEQ ID NO: 92 - DNA, 25 bp, Artificial Sequence
  Primers/Probes Sequences
tatatgtgctcaggttcccagttga

SEQ ID NO: 93 - DNA, 25 bp, Artificial Sequence
  Primers/Probes Sequences
agaagaattattttaggatgtggca

SEQ ID NO: 94 - DNA, 25 bp, Artificial Sequence
  Primers/Probes Sequences
gtytaaaacaaaatagccacagcgt

SEQ ID NO: 95 - DNA, 25 bp, Artificial Sequence
  Primers/Probes Sequences
ggcaaaaagcccacttagcctggct

SEQ ID NO: 96 - DNA, 28 bp, Artificial Sequence
  Primers/Probes Sequences
ttttgactaggaaacagctatgaccatg

SEQ ID NO: 97 - DNA, 25 bp, Artificial Sequence
  Primers/Probes Sequences
ctgcggcaaggcaagacgctcagag

SEQ ID NO: 98 - DNA, 25 bp, Artificial Sequence
  Primers/Probes Sequences
gcgcaggtcacgaggcagtgcgcga

SEQ ID NO: 99 - DNA, 28 bp, Artificial Sequence
  Primers/Probes Sequences
ttttgactaggaaacagctatgaccatg

SEQ ID NO: 100 - DNA, 25 bp, Artificial Sequence
  Primers/Probes Sequences
ggcagtgcgcgagggagctccaggc

SEQ ID NO: 101 - DNA, 25 bp, Artificial Sequence
  Primers/Probes Sequences
ttgggcacagccgagctgctctgcc

SEQ ID NO: 102 - DNA, 25 bp, Artificial Sequence
  Primers/Probes Sequences
catcccggaatatgtgaacagcctg

SEQ ID NO: 103 - DNA, 28 bp, Artificial Sequence
  Primers/Probes Sequences
ttttgactaggaaacagctatgaccatg

SEQ ID NO: 104 - DNA, 25 bp, Artificial Sequence
  Primers/Probes Sequences
acagcctgggagcgctgcgggaatg

SEQ ID NO: 105 - DNA, 13 bp, Artificial Sequence
  Primers/Probes Sequences
gagaaacgggaca

SEQ ID NO: 106 - DNA, 14 bp, Artificial Sequence
  Primers/Probes Sequences
gagaaatgggacaa

SEQ ID NO: 107 - DNA, 14 bp, Artificial Sequence
  Primers/Probes Sequences
ggatcgttccaata

SEQ ID NO: 108 - DNA, 15 bp, Artificial Sequence
  Primers/Probes Sequences
ggatcattccaataa

SEQ ID NO: 109 - DNA, 14 bp, Artificial Sequence
  Primers/Probes Sequences
ctgaattctcacct

SEQ ID NO: 110 - DNA, 15 bp, Artificial Sequence
  Primers/Probes Sequences
ctgaattatcacctt

SEQ ID NO: 111 - DNA, 11 bp, Artificial Sequence
  Primers/Probes Sequences
ccccgcctagc

SEQ ID NO: 112 - DNA, 12 bp, Artificial Sequence
  Primers/Probes Sequences
cccccacctagc

SEQ ID NO: 113 - DNA, 11 bp, Artificial Sequence
  Primers/Probes Sequences
cacgccggcct

SEQ ID NO: 114 - DNA, 11 bp, Artificial Sequence
  Primers/Probes Sequences
cacgctggcct

SEQ ID NO: 115 - DNA, 11 bp, Artificial Sequence
  Primers/Probes Sequences
ggaccgccgtc

SEQ ID NO: 116 - DNA, 12 bp, Artificial Sequence
  Primers/Probes Sequences
ggaccaccgtct

SEQ ID NO: 117 - DNA, 13 bp, Artificial Sequence
  Primers/Probes Sequences
accagtttcctct

SEQ ID NO: 118 - DNA, 13 bp, Artificial Sequence
  Primers/Probes Sequences
accagtctcctct

SEQ ID NO: 119 - DNA, 14 bp, Artificial Sequence
  Primers/Probes Sequences
accaaggaaaagtt

SEQ ID NO: 120 - DNA, 15 bp, Artificial Sequence
  Primers/Probes Sequences
accaagtaaaagttc

SEQ ID NO: 121 - DNA, 14 bp, Artificial Sequence
  Primers/Probes Sequences
atgactaccacaca

SEQ ID NO: 122 - DNA, 14 bp, Artificial Sequence
  Primers/Probes Sequences
atgactatcacaca

SEQ ID NO: 123 - DNA, 13 bp, Artificial Sequence
  Primers/Probes Sequences
agcagctggtaag

SEQ ID NO: 124 - DNA, 12 bp, Artificial Sequence
  Primers/Probes Sequences
gcagccggtaag

SEQ ID NO: 125 - DNA, 11 bp, Artificial Sequence
  Primers/Probes Sequences
accgtcccggg

SEQ ID NO: 126 - DNA, 11 bp, Artificial Sequence
  Primers/Probes Sequences
accgtcctggg

SEQ ID NO: 127 - DNA, 25 bp, Artificial Sequence
  Primers/Probes Sequences
tgcctggtgctggtggaacccctgc

SEQ ID NO: 128 - DNA, 24 bp, Artificial Sequence
  Primers/Probes Sequences
ggcaggcagcgccggacagaccgc

SEQ ID NO: 129 - DNA, 25 bp, Artificial Sequence
  Primers/Probes Sequences
tggtgctggtggaacccctgcacag

SEQ ID NO: 130 - DNA, 28 bp, Artificial Sequence
  Primers/Probes Sequences
ttttgactaggaaacagctatgaccatg

SEQ ID NO: 131 - DNA, 25 bp, Artificial Sequence
  Primers/Probes Sequences
cgagcagttggccaagatctgaccg

SEQ ID NO: 132 - DNA, 25 bp, Artificial Sequence
  Primers/Probes Sequences
ccagctgtgtctccggaaactcgag

SEQ ID NO: 133 - DNA, 25 bp, Artificial Sequence
  Primers/Probes Sequences
agttggccaagatctgaccgtgatg

SEQ ID NO: 134 - DNA, 28 bp, Artificial Sequence
  Primers/Probes Sequences
ttttgactaggaaacagctatgaccatg

SEQ ID NO: 135 - DNA, 25 bp, Artificial Sequence
  Primers/Probes Sequences
tgctttgtcggtgctgatctcagtg

SEQ ID NO: 136 - DNA, 25 bp, Artificial Sequence
  Primers/Probes Sequences
ttactgatgaccatcctcaggttgc

SEQ ID NO: 137 - DNA, 28 bp, Artificial Sequence
  Primers/Probes Sequences
ttttgactaggaaacagctatgaccatg

SEQ ID NO: 138 - DNA, 25 bp, Artificial Sequence
  Primers/Probes Sequences
catcctcaggttgcctaaagctgtc

SEQ ID NO: 139 - DNA, 25 bp, Artificial Sequence
  Primers/Probes Sequences
ctacccgagggaacggaggataaag

SEQ ID NO: 140 - DNA, 25 bp, Artificial Sequence
  Primers/Probes Sequences
agccattctgctcagcccaagtagg

SEQ ID NO: 141 - DNA, 28 bp, Artificial Sequence
  Primers/Probes Sequences
ttttgactaggaaacagctatgaccatg

SEQ ID NO: 142 - DNA, 25 bp, Artificial Sequence
  Primers/Probes Sequences
ccccaccttgtccttacccagcatg

SEQ ID NO: 143 - DNA, 25 bp, Artificial Sequence
  Primers/Probes Sequences
cttgtggatgttctggccaagtttc

SEQ ID NO: 144 - DNA, 25 bp, Artificial Sequence
  Primers/Probes Sequences
tacagcatagtaggtgttgaacacg

SEQ ID NO: 145 - DNA, 25 bp, Artificial Sequence
  Primers/Probes Sequences
tgttctggccaagtttcaactctgc

SEQ ID NO: 146 - DNA, 28 bp, Artificial Sequence
  Primers/Probes Sequences
ttttgactaggaaacagctatgaccatg

SEQ ID NO: 147 - DNA, 25 bp, Artificial Sequence
  Primers/Probes Sequences
ttacccacacgctatttctttgcag

SEQ ID NO: 148 - DNA, 25 bp, Artificial Sequence
  Primers/Probes Sequences
tgtctagtttcttaccggcaggtac

SEQ ID NO: 149 - DNA, 28 bp, Artificial Sequence
  Primers/Probes Sequences
ttttgactaggaaacagctatgaccatg

SEQ ID NO: 150 - DNA, 25 bp, Artificial Sequence
  Primers/Probes Sequences
cggcaggtacttggctcccccgacg

SEQ ID NO: 151 - DNA, 25 bp, Artificial Sequence
  Primers/Probes Sequences
attccccacagctccatcatgggct

SEQ ID NO: 152 - DNA, 25 bp, Artificial Sequence
  Primers/Probes Sequences
cccacatgccattgccggctccgac

SEQ ID NO: 153 - DNA, 25 bp, Artificial Sequence
  Primers/Probes Sequences
ctccatcatgggctacaacttcagc

SEQ ID NO: 154 - DNA, 28 bp, Artificial Sequence
  Primers/Probes Sequences
ttttgactaggaaacagctatgaccatg

SEQ ID NO: 155 - DNA, 25 bp, Artificial Sequence
  Primers/Probes Sequences
ggattggcttccaggtcctcctcag

SEQ ID NO: 156 - DNA, 25 bp, Artificial Sequence
  Primers/Probes Sequences
ctgacctgtcaggagaccagacatg

SEQ ID NO: 157 - DNA, 25 bp, Artificial Sequence
  Primers/Probes Sequences
ggcttccaggtcctcctcagcattg

SEQ ID NO: 158 - DNA, 28 bp, Artificial Sequence
  Primers/Probes Sequences
ttttgactaggaaacagctatgaccatg

SEQ ID NO: 159 - DNA, 25 bp, Artificial Sequence
  Primers/Probes Sequences
aaggatttctgttgagatactgtcg

SEQ ID NO: 160 - DNA, 25 bp, Artificial Sequence
  Primers/Probes Sequences
catgaggtgctttccatattttaag

SEQ ID NO: 161 - DNA, 25 bp, Artificial Sequence
  Primers/Probes Sequences
tgtcgttttgacacacaatatttcg

SEQ ID NO: 162 - DNA, 28 bp, Artificial Sequence
  Primers/Probes Sequences
ttttgactaggaaacagctatgaccatg

SEQ ID NO: 163 - DNA, 14 bp, Artificial Sequence
  Primers/Probes Sequences
tgagctctaagtac

SEQ ID NO: 164 - DNA, 14 bp, Artificial Sequence
  Primers/Probes Sequences
tgagctgtaagtac

SEQ ID NO: 165 - DNA, 12 bp, Artificial Sequence
  Primers/Probes Sequences
tggcttgggctt

SEQ ID NO: 166 - DNA, 13 bp, Artificial Sequence
  Primers/Probes Sequences
tggctttggcttc

SEQ ID NO: 167 - DNA, 13 bp, Artificial Sequence
  Primers/Probes Sequences
aactgcgccaagt

SEQ ID NO: 168 - DNA, 14 bp, Artificial Sequence
  Primers/Probes Sequences
aactgcaccaagtt

SEQ ID NO: 169 - DNA, 13 bp, Artificial Sequence
  Primers/Probes Sequences
gttgctgtctgat

SEQ ID NO: 170 - DNA, 13 bp, Artificial Sequence
  Primers/Probes Sequences
gttgctctctgat

SEQ ID NO: 171 - DNA, 14 bp, Artificial Sequence
  Primers/Probes Sequences
tccaatcgaaagga

SEQ ID NO: 172 - DNA, 14 bp, Artificial Sequence
  Primers/Probes Sequences
ccaatccaaaggaa

SEQ ID NO: 173 - DNA, 14 bp, Artificial Sequence
  Primers/Probes Sequences
ccaatcaaaaggaa

SEQ ID NO: 174 - DNA, 14 bp, Artificial Sequence
  Primers/Probes Sequences
ctgtgcacataagt

SEQ ID NO: 175 - DNA, 13 bp, Artificial Sequence
  Primers/Probes Sequences
ctgtgcccataag

SEQ ID NO: 176 - DNA, 13 bp, Artificial Sequence
  Primers/Probes Sequences
tgcttgataccgt

SEQ ID NO: 177 - DNA, 13 bp, Artificial Sequence
  Primers/Probes Sequences
gtgcttcataccg

SEQ ID NO: 178 - DNA, 17 bp, Artificial Sequence
  Primers/Probes Sequences
ccatcgtgatagctctc

SEQ ID NO: 179 - DNA, 14 bp, Artificial Sequence
  Primers/Probes Sequences
aaacaggtttgctc

SEQ ID NO: 180 - DNA, 14 bp, Artificial Sequence
  Primers/Probes Sequences
aaacagctttgctc

SEQ ID NO: 181 - DNA, 25 bp, Artificial Sequence
  Primers/Probes Sequences
ttccgcacgcctctctccatgtgca

SEQ ID NO: 182 - DNA, 25 bp, Artificial Sequence
  Primers/Probes Sequences
ttctccatgtgcagtaggaaggatg

SEQ ID NO: 183 - DNA, 25 bp, Artificial Sequence
  Primers/Probes Sequences
tttttgccttcacctacgagcgccg

SEQ ID NO: 184 - DNA, 25 bp, Artificial Sequence
  Primers/Probes Sequences
ttttttctacgagcgccggccccag

SEQ ID NO: 185 - DNA, 25 bp, Artificial Sequence
  Primers/Probes Sequences
ttgaagctgaggttcactgcggtgc

SEQ ID NO: 186 - DNA, 25 bp, Artificial Sequence
  Primers/Probes Sequences
agcccaagctctaccttgcctcccg

SEQ ID NO: 187 - DNA, 26 bp, Artificial Sequence
  Primers/Probes Sequences
aaaccattttttcctgatacctcttg

SEQ ID NO: 188 - DNA, 33 bp, Artificial Sequence
  Primers/Probes Sequences
ttaagaaagtatgtgaaactgacacagccaagg

SEQ ID NO: 189 - DNA, 25 bp, Artificial Sequence
  Primers/Probes Sequences
ataacacttgtccacaggggtgttg

SEQ ID NO: 190 - DNA, 25 bp, Artificial Sequence
  Primers/Probes Sequences
gttgtaaccgagtgctggggattcc

SEQ ID NO: 191 - DNA, 25 bp, Artificial Sequence
  Primers/Probes Sequences
ttctctaccctcggccacctcactg

SEQ ID NO: 192 - DNA, 25 bp, Artificial Sequence
  Primers/Probes Sequences
ttcaatggtggtgttctggagcctg

SEQ ID NO: 193 - DNA, 25 bp, Artificial Sequence
  Primers/Probes Sequences
tttacccgttctgctaaaaccaagg

SEQ ID NO: 194 - DNA, 25 bp, Artificial Sequence
  Primers/Probes Sequences
ttcctggtggcaggccctggtgagc

SEQ ID NO: 195 - DNA, 26 bp, Artificial Sequence
  Primers/Probes Sequences
tttttcagccctcccagagccccctg

SEQ ID NO: 196 - DNA, 25 bp, Artificial Sequence
  Primers/Probes Sequences
ataattaatggctttcattattggg

SEQ ID NO: 197 - DNA, 33 bp, Artificial Sequence
  Primers/Probes Sequences
tttcggaaaatttgcagcctgacaatgtgatag

SEQ ID NO: 198 - DNA, 33 bp, Artificial Sequence
  Primer/Probe Sequences
ttaattaatggctttcattattgggtatatttg

SEQ ID NO: 199 - DNA, 25 bp, Artificial Sequence
  Primers/Probes Sequences
ccacatgccattgccggctccgacg

SEQ ID NO: 200 - DNA, 24 bp, Artificial Sequence
  Primers/Probes Sequences
ttttgccgctccgacggtatcaag